SEQUENCE LISTING (5 sequences)

SEQ ID NO: 1 - protein, 159 aa, Artificial sequence
  PEPTIDE (1)..(159): Synthetic primer for detecting CYP specific to candicidin
LLLIAGHETTANNIGLGVVTLLSHREWIGDDRLVEELLRLHSVADMVALRVAVDDVEIAGQTIRKGEGIVPLLASANHDTEAFGCPHAFNPERTERRHVAFGYGVHQCLGQNLVRVEMEIAYRKLFERIPELRLAVPEDQLAYKYDGILFGLHELPVRW

SEQ ID NO: 2 - protein, 159 aa, Artificial Sequence
  PEPTIDE (1)..(159): Synthetic primer for detecting CYP specific to pimaricin
LLLIAGHETTANNIALGVVTLLANPQWIGDDRAVEETLRFHSVADLVSLRVAVQDVEIAGQLIKAGEGIVPLVAAANHDENAFECPHAFDPSRSARHHVAFGYGVHQCLGQNLVRIEMEVAYRKLFERIPNLELAVPTDGLDIKYDGVLYGLNELPVRW

SEQ ID NO: 3 - protein, 160 aa, Artificial sequence
  PEPTIDE (1)..(160): Synthetic primer for detecting CYP specific to amphotericin
LLLIAGHETTANNIGLGVVQLLTNPQWIGDDRIVEEMLRYYSVADLVSFRVAVEDVEIGGQLIKAGEGIVPLIAAANHDGSVFDKPEEFNPERSARSHVAFGYGVHQCLGQNLVRVEMEIAYRTLFERIPTLELAVPVEELPLKYDGVLFGLHELPVTWS

SEQ ID NO: 4 - protein, 160 aa, Artificial sequence
  PEPTIDE (1)..(160): Synthetic primer for detecting CYP specific to nystatin
LLLIAGHETTANNIGLGVVQLLTNPRWIGDDRIVEELLRYYSVADLVAFRVAVEDVEIGGQLIRAGEGIVPLIAAANHDATAFAAPSEFDPERSARSHVAFGYGVHQCLGQNLVREEMDIAYRTLFARIPSLTLAVPVEELPLKYDGVLFGLHELPVTWK

SEQ ID NO: 5 - DNA, 374 bp, Artificial sequence
  CYP gene isolated from P. autotrophica
tggatcggcgacgaccgcgtcgtcgaggagctgctgcgctactactcggtggccgacctggtcgcgttccgggtcgcgctggccgacgtcgagatcggcgggcgcacgatccgcgcgggcgagggcatcctgccgctgctggccgccgccaaccacgacgacgacgccttcgacggcgccggcgcgttcgacccggaacgctccgcgcgctcgcacgtggccttcggctacggcgtgcaccagtgcctgggccagaacctggtgcggctggagatggagatcgcctaccgcacgctgttcgaccggatcccgacgctgcgcctggcggtgcccgccgatgacctgcgcgtgaagtacgacggcgtgttgttcgg

(19) United States
(12) Patent Application Publication
(10) Pub. No.: US 2010/0003669 A1
(43) Pub. Date: Jan. 7, 2010

(54) Primer for Detection of Cytochrome P450 Hydroxylase Specific to Polyene

(75) Inventors: Kyuboem Han, Daejeon (KR); Sangnyun Kim, Seoul (KR); Hyunjoo Park, Seoul (KR); JiSeon Myeong, Incheon (KR); Namsil Park, Gyeonggi-do (KR); Engsoo Kim, Seoul (KR)

Correspondence Address: THE NATH LAW GROUP, 112 South West Street, Alexandria, VA, 22314, US

(73) Assignee: HANSON BIOTECH CO., LTD., Daejeon (KR)

(21) Appl. No.: 11/664,424

(22) PCT Filed: Sep. 26, 2005
(86) PCT No.: PCT/KR05/03185

§ 371 Date: Jun. 14, 2007

(30) Foreign Application Priority Data
Oct. 4, 2004 (KR) 10-2004-0078613

(51) Int. Cl. C12Q 1/68; C07H 21/04

(52) U.S. Cl. 435/6; 536/24.33

(57) ABSTRACT

The present invention relates to a primer for detection of cytochrome P450 hydroxylase specific to polyene and a method for detecting a strain of bacteria which produces polyene, by using the primer. More particularly, the present invention is directed in a forward degenerate primer of following sequence (I) and a reverse degenerate primer of following sequence (II), which are complementary to conserved region which resides in the polyene-specific CYP (cytochrome P450 hydroxylose) encoding sequences: (I) 5′-TGGATCGGCGACGACCGSVYCGT-3′ and (II) 5′-CCGWASAGSAYSCCGTCOTACTT-3′, wherein S indicated Guanine or Cytosine, V indicates Adrenine, Guanine or Cytosine, Y indicates Thymine or Cytosine and W indicates Thymine or Adenine.

### TECHNICAL FIELD

The present invention relates to a primer for detection of cytochrome P450 hydroxylase specific to polyene and a method for detecting a strain of bacteria which produces polyene, by using the primer. More particularly, the present invention is directed to a forward degenerate primer of following sequence (I) and a reverse degenerate primer of following sequence (II), which are complementary to conserved region which resides in the polyene-specific CYP (cytochrome P450 hydroxylase) encoding sequences;

(I): 5′-TGGATCGGCGACGACCGSVYCGT-3′
(II): 5′-CCGWASAGSAYSCCGTCGTACTT-3′

In the above sequence, S indicates Guanine or Cytosine, V indicates Adenine, Guanine or Cytosine, Y indicates Thymine or Cytosine and W indicates Thymine or Adenine.

### BACKGROUND ART

In this specification, polyene is a general name of organic compounds which contain several carbon-carbon double bonds in their chemical formula. Generally, a polyene antibiotic is called a polyenemacrolide. In other words, a polyene antibiotic means an antibiotic which contains at least three conjugated double bond among the macrolide antibiotics which have lactone chemical structures of large ring shape.

A polyene antibiotic has a macrolactone ring structure which consists of 20 to 40 carbons and comprises approximately four (4) to seven (7) conjugated double bonds in the molecule. Also, polyene antibiotic has an antifungal activity. The polyene antibiotic absorb UV of specific wave due to the conjugated double bonds and is classified into tetraene, pentaene, hexaene, heptaene etc. according to the number of conjugated double bond. Yeast comprising a candida, mold and trichomonas etc. are available as a polyene antibiotic and amphotericin B, pimaricin and pentamycin etc. is clinically used.

The majority of polyene antibiotics which have been known up to now, are generally biosynthesized by actinomycetes. Actinomycetes are gram-positive soil microorganisms which form spores and make a mycelium of filament type and cause particular form differentiation according to the change of environment. The actinomycete is a strain which produces various kinds of material which has useful physiological activity, such as antibiotic or anticancer substance.

The polyene compounds is known to have predominant antifungal activity for fungus such as mold and yeast by bonding with sterol of cell membrane of fungus and forming a channel which causes metabolic trouble of leaking intracellular component such as K⁺, Mg²⁺ etc. Therefore, the polyene compounds do not function to microbes which do not have sterol.

Even though polyene compounds are limitedly employed as medicine due to the side effect such as strong toxic, it has been needed to develop novel and improved polyenemacrolide antibiotics owing to their predominant antifungal activity.

The study for actinomycetes which synthesize typical polyene compounds such as nystatin, amphotericin, pimaricin, candicidin etc, is actively progressing. Also, base sequences of gene cluster related to the polyene biosynthesis of actinomycetes, are completely decoded recently.

This gene cluster comprises the genes of PKS (polyketide synthase), transport, regulator, post-polyketide modification which are commonly needed to the polyene biosynthesis.

Generally, in the biosynthesis of the polyene compounds, polyketide biosynthesis which is repetitive carbon chain reaction, is preceded and subsequently post-polyketide modification is performed. In these processes, site-specific hydroxylation reaction by CYP (cytochrome P540 hydroxylase), is essential to make the polyene have antifungal activity.

CYP comprises conserved regions of oxygen binding site and heme ligand pocket, and engage in activation of various precursor antibiotics and detoxification of low re-solvability materials. Also, CYP is an enzyme which catalyzes various oxidation reactions by employing various compounds such as polyketide, fatty acid, steroid as a substrate.

The gene of polyene-specific CYP have additional conserved region in addition to conserved sequences of oxygen binding site and heme ligand pocket which exist in gene of general CYP. The base sequence of this additional conserved region is illustrated in FIG. 2.

For the development of novel polyene macrolid, it is needed to develop an effective method for identifying a strain which can produce the polyene.

While the genes related to the polyene biosynthesis have been studied, it has been attempted to identify a novel strain which can produce polyene by detecting the existence of specific gene essential to polyene biosynthesis, contrary to the conventional strain selection method. Such method is called as genomics-based in silico drug development method.

Genomics-based in silico drug development method is to search a useful microorganism which enable to synthesize antibiotics or useful materials from the gene information of microorganisms instead of prior method which isolate microorganism which can produce polyene compounds and detect corresponding genes from isolated microorganism. This method can identify microorganisms which cannot synthesize antibiotics or useful materials since various conditions is not optimized in despite of having the ability to synthesize antibiotics or useful materials.

Therefore, in this technical field, it has been eagerly requested to develop genomics-based in silico technology, which detects a strain which has antifungal and antivirus activity by obtaining information of polyene-specific CYP gene and amplifying the CYP gene in a sample.

### DISCLOSURE OF INVENTION

### Technical Problem

Therefore, the primary object of the present invention is to provide a forward degenerate primer of following sequence (I) and a reverse degenerate primer of following sequence (II), which are complementary to conserved region of polyene-specific CYP (cytochrome P450 hydroxylase) encoding gene sequences and can be employed effectively for the amplification of the conserved region:

(I): 5′-TGGATCGGCGACGACCGSVYCGT-3′
(II): 5′-CCGWASAGSAYSCCGTCGTACTT-3′

In the above sequences, S indicates Guanine or Cytosine, V indicates Adenine, Guanine or Cytosine, Y indicates Thymine or Cytosine and W indicates Thymine or Adenine.

The degenerated primers of the present invention are employed for selective amplification and detection of specific portion of CYP genes in a bacterial strain. The primers combines with the conserved region of polyene-specific CYP gene and thus, can be used as a PCR primer for detection of the strain capable of polyene biosynthesis.

Another object of the present invention is to provide a method for detecting a strain of bacteria which can produce polyene, which comprises steps of i) adding degenerate primers which are complementary to conserved region of polyene-specific CYP encoding sequences, four (4) kinds of deoxynucleotides and DNA polymerase to gene of a bacterial strain; and ii) identifying whether or not the bacterial strain is capable of producing polyene by examining whether or not DNA replication is occurred.

### Technical Solution

The above mentioned object of the present invention can be achieved by providing a forward degenerate primer of following sequence (I) and a reverse degenerate primer of following sequence (II), which are complementary to conserved region of polyene-specific CYP (cytochrome P450 hydroxylase) encoding sequences:

(I) 5′-TGGATCGGCGACGACCGSVYCGT-3′;
and
(II) 5′-CCGWASAGSAYSCCGTCGTACTT-3′

In the above sequences, S indicates Guanine or Cytosine, V indicates Adenine, Guanine or Cytosine, Y indicates Thymine or Cytosine and W indicates Thymine or Adenine.

A degenerate primer is a DNA fragment in which some base positions contain more than one possible base. This degenerate primers can be used for amplifying various similar sequences.

The degenerate forward primer PEH-1 of the present invention is 5′-TGGATCGGCGACGACCGSVYCGT-3′; wherein S indicates G or C, V indicates A, G or C, Y indicates T or C. The degenerate reverse primer PEH-2 of the present invention is 5′-CCGWASAGSAYSCCGTCGTACTT-3′; wherein W indicates T or A, S indicates G or C, Y indicates T or C.

To produce degenerate PCR primers for cloning polyene-specific CYP gene, the sequences of CYP genes related to polyene biosynthesis should be compared with each other and analyzed. The sequences of CYP genes related to polyene biosynthesis are illustrated in FIG. 2.

The another object of the present invention can be achieved by providing a method for detecting a strain of bacteria which produces polyene, comprising the steps of i) adding degenerate primers which are complementary to conserved region of polyene-specific CYP encoding sequences, four(4) kinds of deoxynucleotides; deoxyadenosine 5′-triphosphate(dATP), deoxycytidine 5′-triphosphate(dCTP), deoxyguanosine 5′-triphosphate(dGTP), deoxythymidine 5′-triphosphate(dTTP), and DNA polymerase to gene of a bacterial strain; and ii) identifying whether or not the bacterial strain is capable of producing polyene by examining whether or not DNA replication is occurred.

The amplification of conserved region of polyene-specific CYP encoding gene, is performed by using the primer of the present invention. Also, it can be known by performing electrophoresis whether or not polyene-specific CYP encoding gene exists in a bacterial strain.

The present inventors detected polyene-specific CYP and actinomycetes which contain polyene biosynthesis gene, by performing PCR using the degenerate primers of the present invention. After the PCR, an amplified gene fragment is detected at approximately 350 bp site from the PCR product obtained from the strain which contains polyene biosynthesis gene. However, the gene fragment at approximately 350 bp site, is not detected from the PCR product obtained from the strain which does not contain polyene biosynthesis gene.

### Advantageous Effects

As mentioned above, a novel polyene-specific CYP gene can be detected by carrying out PCR using the degenerate primers of the present invention, from the gene of bacterial strains, such as P. autotrophica and S. benihana of which capability for the production of polyene-specific CYP had not yet been known through the conventional research methods.

### BRIEF DESCRIPTION OF THE DRAWINGS

FIG. 1 shows the chemical structures of polyene antibiotic which have antifungal and antiviral activity.

FIG. 2 shows the conserved regions of amino acid sequence of polyene-specific CYP.

FIG. 3 shows the result of agarose gel electrophoresis of PCR products.

FIG. 4 shows the base sequence of Pseudonorcardia autotrophica which is analyzed by cloning the PCR fragment of approximately 30 bp which is estimated as a polyene-specific CYP.

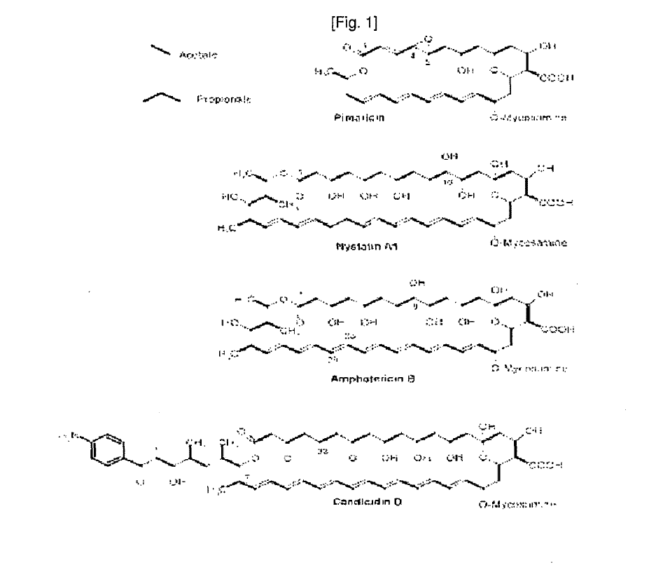

### BEST MODE FOR CARRYING OUT THE INVENTION

Practical and preferred embodiments of the present invention will be illustrated in more detail in the following Examples. The present invention is not limited to them and can allow various modifications and alterations performed by those skilled in the art.

### Example 1

### The Production of Degenerate Primer

The CYPs of actinomycetes have analogous amino acid sequences and the similarity is very high especially in oxygen binding site and heme ligand pocket. Also, in addition to these two regions, another highly-conserved region exists in CYP proteins which participates in polyene biosynthesis.

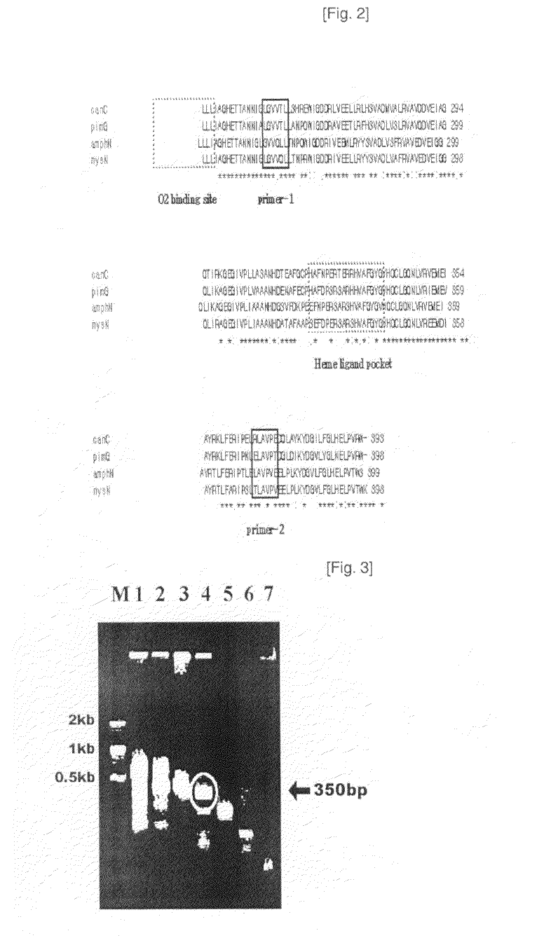

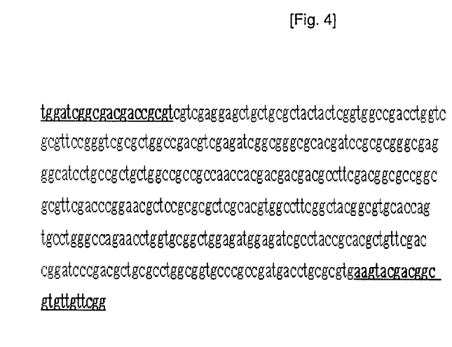

To detect polyene-specific CYP, degenerate primers were prepared by means of the information of the amino acid sequences of highly-conserved region of CYP protein related to only polyene biosynthesis. The high-conversed region employed to prepare the degenerate primer of the present invention, is indicated in FIG. 2 as a solid line.

### Example 2

### The Method for Detection of Polyene-Specific CYP and for Cultivation of a Bacterial Strain

Three (3) species of actinomycetes of Streptomyces coelicolor M145, Streptomyces avermitilis (ATCC31267) and Streptomyces peucetius (ATCC29050) which do not contain the polyene-biosynthesis gene, two (2) species of bacterial strains of Streptomyces nodosus (KCTC 9035) and Streptomyces noursei (KCTC 1083) which produce polyene, and two (2) species of rare actinmycetes of Pseudonocardia autotrophica (KCTC 9441) and Sebekia benihana (KCTC 9660) which whether or not they can produce polyene is unknown, were used as the strain for the detection of polyene-specific CYP.

Each strain was grown into mycelium type in R2YE liquid medium and frozen to -20° C. and used as type of 20% glycerol stock. The stock was inoculated in YEME liquid medium (Sucrose 340g, Yeast extract 3g, Bacto-peptone 5g, Malt extract 3g, Clucose 10g, D.W. 1L) to obtain the strain used to isolate respective genomic DNAs. The respective strains were obtained by using a centrifugal separator (800 rpm) after cultured for five days at 28° C.

### Example 3

### The performance of PCR

A pair of degenerate primers produced in the Example 1 of the present invention, Taq polymerase, 2′-Deoxyadenosine 5′-triphosphate (dATP), 2′-Deoxycytidine 5′-triphosphate (dCTP), 2′-Deoxyguanosine 5′-triphosphate (dGTP) and 2′-Deoxythymidine 5′triphosphate (dTTP), were added to the actinomycetes cultured in Example 2. And then, the actinomycetes were denatured for 30 second at 96° C. by using Rapid Thermocycler (Idaho technology, USA) and annealing the primer for 30 second at 40° C. and extended for 35 second at 72° C. Said cycle was repeated thirty times in PCR.

### MODE FOR THE INVENTION

### Example 4

### The Result of Polyene-Specific CYP Detection

After the electrophoresis of PCR product was performed, amplified gene fragment was detected at 350 bp site in Streptomyces nodosus and Streptomyces noursei which reported as polyene producing strains. However, amplified DNA fragment of expected size was not detected in Streptomyces coelicolor, Streptomyces avermitilis and Streptomyces peucetius which did not contain polyene biosynthesis gene.

The above result indicates that polyene-specific CYP gene can be detected selectively by using degenerate primers of the present invention in despite of existence of various species of analogous CYP in the majority of actinmycetes.

Particularly, an amplified gene fragment which can be estimated as a polyene-specific CYP, was detected at approximately 350 bp site from the PCR product of gene of Pseudonocardia autotrophica, a rare actinmycete of which capability for the biosynthesis of polyene, is unknown.

### Example 5

### The Analysis of Amplified Sequence which is Presumed as Polyene-Specific CYP in Pseudonocardia autotrophica

An amplified fragment of approximately 350 bp of Pseudonocardia autotrophica, was cloned and analyzed by using pGEMT-easy vector(Promega, USA). The result indicated that the amplified gene fragment is a novel polyene-specific CYP gene highly analogous to the conventional polyene-specific CYP gene which had been known to the public.

### INDUSTRIAL APPLICABILITY

The conventional research methods were very laborious and time-consuming process since the conventional steps for culture a strain and for inspection of the formation of polyene compound were required for the examination of the ability for production of polyene compounds of a strain. Further, more time and laborious efforts were required for the examination of ability for the production of polyene compound of a strain of which culture condition is intricate and difficult.

The capability for the production of polyene compounds of the various kind of strains, can be examined more quickly and simply by employing degenerate primers and detection method of the present invention.

### SEQUENCE LISTING

File attachment HSPA0403.APP

1. A forward degenerate primer of following sequence (I) and a reverse degenerate primer of following sequence (II) which are complementary to conserved region of polyene-specific CYP (cytochrome P450 hydroxylase) encoding sequences;
(I) 5′-TGGATCGGCGACGACCGSVYCGT-3′
(II) 5′-CCGWASAGSAYSCCGTCGTACTT-3′
in the above sequences, S indicates Guanine or Cytosine, V indicates Adenine, Guanine or Cytosine, Y indicates Thymine or Cytosine and W indicates Thymine or Adenine.

2. A method for the detection of bacteria which produce polyene compound, which comprises steps for;
i) adding degenerate primers which are complementary to a conserved region of polyene-specific CYP encoding sequences, four (4) kinds of deoxynucleotides and DNA polymerase to gene of a bacteria; and
ii) examining the replication of said conserved region.

3. The method according to claim 2, wherein the degenerate primers of said step i) are a forward degenerate primer of following sequence (I) and a reverse degenerate primer of following sequence (II):
(I) 5′-TGGATCGGCGACGACCGSVYCGT-3′
(II) 5′-CCGWASAGSAYSCCGTCGTACTT-3′
In the above sequences, S indicates Guanine or Cytosine, V indicates Adenine, Guanine or Cytosine, Y indicates Thymine or Cytosine and W indicates Thymine or Adenine.

4. The method according to claim 2, wherein the step ii) is performed through electrophoresis process.

5. The method according to claim 2, wherein the step ii) is performed through PCR process wherein deoxynucleotide labeled with fluorescent material is employed.
